(19) United States
(12) Patent Application Publication
(10) Pub. No.: US 2003/0182067 A1
(43) Pub. Date: Sep. 25, 2003

(54) Biological information management apparatus and memory product

(75) Inventors: Masakazu Asano, Osaka (JP); Rie Monchi, Kyoto-Shi (JP); Hirokazu Genno, Osaka (JP)

Correspondence Address: DARBY & DARBY P.C., Post Office Box 5257, New York, NY, 10150-5257, US

(73) Assignee: SANYO ELECTRIC CO., LTD., Moriguchi City (JP)

(21) Appl. No.: 10/356,648

(22) Filed: Jan. 31, 2003

(30) Foreign Application Priority Data
Jan. 31, 2002 (JP) 2002-024824

(51) Int. Cl. G06F 17/60; G06F 19/00; G01N 33/48; G01N 33/50

(52) U.S. Cl. 702/19; 705/3

(57) ABSTRACT

First criteria and second criteria which are different from the first criteria are calculated, based on the past biological information stored in a biological information database. When biological information is inputted, it is compared with the calculated first criteria or second criteria. If it is judged that the inputted biological information does not meet the first criteria, one category of message data is determined from a plurality of categories. On the other hand, if it is judged that the inputted biological information meets the first criteria but does not meet the second criteria, a category of message data different from the category determined by the first determining means is determined, and a message belonging to the determined category is outputted.

### BACKGROUND OF THE INVENTION

1. Field of the Invention

The present invention relates to a biological information management apparatus for storing inputted biological information in a biological information database and outputting the stored biological information to the outside, and also relates to a memory product for causing a computer to function as a biological information management apparatus. The present invention particularly relates to a biological information management apparatus and a memory product, capable of selecting and outputting optimum message data, according to inputted biological information.

2. Description of the Prior Art

In recent years, the number of people cared for, such as older people who need nursing care, is increasing. Therefore, the development of biological information management apparatuses with the functions to store biological information, such as blood pressure, weight, body temperature or blood sugar, and transmit the stored biological information to a computer in a hospital, etc. is being actively pursed. For example, biological information management apparatuses in the shape of a pet such as a cat and a dog have been released to make the people cared for feel attached to the apparatuses.

However, many of older people are not experienced in operating apparatuses. Thus, there are problems associated with inaccurate inputting of biological information such as body temperature to the biological information management apparatus. One example of such a problem is that a definitely wrong numerical value such as 76° C. was inputted for body temperature. Moreover, the biological information management apparatus stores everyday biological information in a biological information database, calculates an average body temperature, etc., and provides doctors and other staff with such information. However, when clearly erroneous biological information such as the one mentioned above is inputted, the reliability of the biological information database decreases considerably.

Besides, an appropriate range of blood sugar is around 70 to 100. However, when the blood sugar of a person cared for, which exceeds this appropriate value, comes closer to the appropriate value gradually as a result of treatment, the mental burden of this person needs to be lessened by some means.

### BRIEF SUMMARY OF THE INVENTION

The present invention has been made with the aim of solving the above problems, and it is an object of the present invention to provide a biological information management apparatus that sets first criteria for rejecting an inputted numerical value which is clearly different from a real value, and, if a numerical value inputted does not meet the first criteria, warns this fact by voice or a message including characters, and is thereby capable of preventing situations of inputting wrong biological information and creating a database, or sending the wrong biological information to doctors, etc., and also to provide a memory product for causing a computer to function as the biological information management apparatus.

Another object of the present invention is to provide a biological information management apparatus that sets second criteria for judging whether an inputted value, which meets the first criteria and thus is not a clearly abnormal value, is biological information different from everyday biological information of a person cared for, and, if the inputted numerical value does not meet the second criteria, is able to output message data indicating this fact, and also to provide a memory product for causing a computer to function as the biological information management apparatus.

Still another object of the present invention is to provide a biological information management apparatus that sets generally appropriate values of biological information of people cared for as third criteria, and, if an inputted numerical value does not meet the third criteria, outputs optimum message data according to a change in the biological information of a person cared for, and is thereby capable of lessening the mental burden of the person cared for, and also to provide a memory product for causing a computer to function as the biological information management apparatus.

Yet another object of the present invention is to provide a biological information management apparatus which, if a voice signal or an operation signal was inputted within a predetermined time after outputting message data, outputs message data or the past biological information according to the signal, and is thereby capable of immediately giving information to a person cared for, who is interested in the biological information, by the output of the message data.

A biological information management apparatus according to the present invention is a biological information management apparatus for storing inputted biological information in a biological information database and outputting the stored biological information to the outside, and characterized by comprising: a message database storing message data classified into a plurality of categories; means for calculating first criteria and second criteria which are different from the first criteria, based on past biological information stored in the biological information database; means for judging whether or not biological information inputted meets the calculated first criteria or second criteria by comparing the inputted biological information with the first criteria or the second criteria; first determining means for determining a category of message data if it is judged that the inputted biological information does not meet the first criteria; second determining means for determining a category of message data different from the category determined by the first determining means if it is judged that the inputted biological information meets the first criteria, but does not meet the second criteria; and a message output unit for outputting message data belonging to the determined category.

The biological information management apparatus according to the present invention is characterized by comprising: means for reading pre-stored third criteria; means for judging whether or not biological information inputted meets the read third criteria by comparing the inputted biological information with the third criteria; means for calculating a change in the biological information, based on the inputted biological information and the past biological information stored in the biological information database if it is judged that the inputted biological information does not meet the third criteria; and third determining means for determining a category of message data, based on the calculated change.

The biological information management apparatus according to the present invention is characterized by comprising: means for judging whether or not a voice signal or an operation signal was inputted within a predetermined time after outputting message data belonging to the category determined by the first determining means, second determining means, or third determining means from the message output unit; means for outputting message data belonging to a predetermined category in the message database from the message output unit if it is judged that a voice signal was inputted; and means for outputting the biological information stored in the biological information database to the outside if it is judged that an operation signal was inputted.

According to the present invention, when biological information such as blood pressure, body temperature, blood sugar, or weight is inputted, the information is stored in the biological information database together with the date information in the biological information database. Further, a message database in which message data such as “Is the inputted item correct?”, “The value is slightly higher today?”, “You look well”, and “Please take a rest” are classified into a plurality of categories according to the contents is prepared.

The biological information management apparatus calculates the first criteria, based on the past biological information stored in the biological information database. In order to prevent apparent input mistakes, values not less than 70% but not more than 130% of the average value of the last ten days, for example, are set as the first criteria. Moreover, in order to give an advice concerning health, the second criteria different from the first criteria are calculated, based on the past biological information stored in the biological information database. Since the first criteria are values not less than 70% but not more than 130% of the average value, for example, a narrower range of values not less than 80% but not more than 120% of the average value than the first criteria is set as the second criteria.

When the biological information is inputted, a judgment is made as to whether or not the inputted biological information meets the calculated first criteria or second criteria by comparing the inputted biological information with the first criteria or the second criteria. If it is judged that the inputted biological information does not meet the first criteria, one category of message data is determined from a plurality of categories. For example, a category including a group of message data that asks for confirmation, such as “Is the inputted item correct?” is determined.

On the other hand, if it is judged that the inputted biological information meets the first criteria but does not meet the second criteria, a category of message data different from the category determined by the first determined means is determined. For example, a category including a group of message data indicating that the inputted biological information differs from the everyday biological information, such as, for example, “The value is slightly higher today” is determined. Then, message data belonging to the determined category is outputted from the message output unit, and thus it is possible to provide a biological information management apparatus capable of preventing situations of inputting wrong biological information and creating a database, or sending the wrong biological information to doctors, etc., and capable of giving appropriate sympathetic words.

Further, according to the present invention, the third criteria which are pre-stored appropriate values are read. The third criteria are generally appropriate values of biological information about blood pressure, body temperature, blood sugar, weight, or the like, and examples of such values are 70 mg/dl to 100 mg/dl for blood sugar, 110 mmHg to 130 mmHg for systolic blood pressure, and 60 mmHg to 80 mmHg for diastolic blood pressure. When biological information is inputted, the inputted biological information is compared with the read third criteria so as to judge whether or not the inputted biological information meets the third criteria. If it is judged that the inputted biological information does not meet the third criteria, a change in the biological information is calculated, based on the inputted biological information and the past biological information stored in the biological information database.

In order to calculate the change, for example, the biological information of the last five days is read and examined whether the biological information is continuously approaching the third criteria or deviating away from the third criteria. Then, a category of message data is determined, based on the calculated change. For example, when the calculation shows a change in which the biological information is approaching the third criteria continuously for five days though the biological information does not meet the third criteria, a category including a group of message data that recognizes the effect of treatment, such as “You are getting better gradually” is selected. Then, message data belonging to this determined category is outputted, and thus it is possible to significantly reduce the mental burden of the person cared for, which is caused by the treatment.

In addition, according to the present invention, a judgment is made as to whether a voice signal or an operation signal was inputted within a predetermined time after outputting message data determined based on the above-mentioned category. If it is judged that a voice signal was inputted, message data belonging to a predetermined category in the message database is outputted from the message output unit. On the other hand, if it is judged that an operation signal was inputted, the biological information stored in the biological information database is outputted to the outside such as the display unit or a server computer connected to the Internet. For example, after the message “The value is slightly higher today” was outputted, if the person cared for gives a voice in response to this message, this voice is recognized, and then message data of sympathetic words is outputted. Whereas if a key operation was made, the biological information is outputted to the display unit or the like. Since the biological information management apparatus is constructed as described above, it is possible to give interesting information immediately to the person cared for, who responded quickly to the message output of the biological information management apparatus. Accordingly, it is also possible to give sympathetic words and significantly reduce the mental burden of the person cared for.

The above and further objects and features of the invention will more fully be apparent from the following detailed description with accompanying drawings.

### BRIEF DESCRIPTION OF THE SEVERAL VIEWS OF THE DRAWINGS

FIG. 1 is a block diagram showing the hardware structure of a biological information management apparatus;

FIG. 2 is an explanatory view showing the record layout of a biological information DB;

FIG. 3 is an explanatory view showing the record layout of a voice DB;.

FIGS. 4A and 4B are a flowchart showing the processing procedure for making judgments based on the first criteria and the second criteria;

FIG. 5 is a flowchart showing the processing procedure to be followed when the third criteria are not met;

FIG. 6 is a flowchart showing the processing procedure after outputting voice data from a voice output unit; and

FIG. 7 is a schematic view showing the abstract of the second embodiment of the present invention.

### DESCRIPTION OF THE PREFERRED EMBODIMENTS

First Embodiment

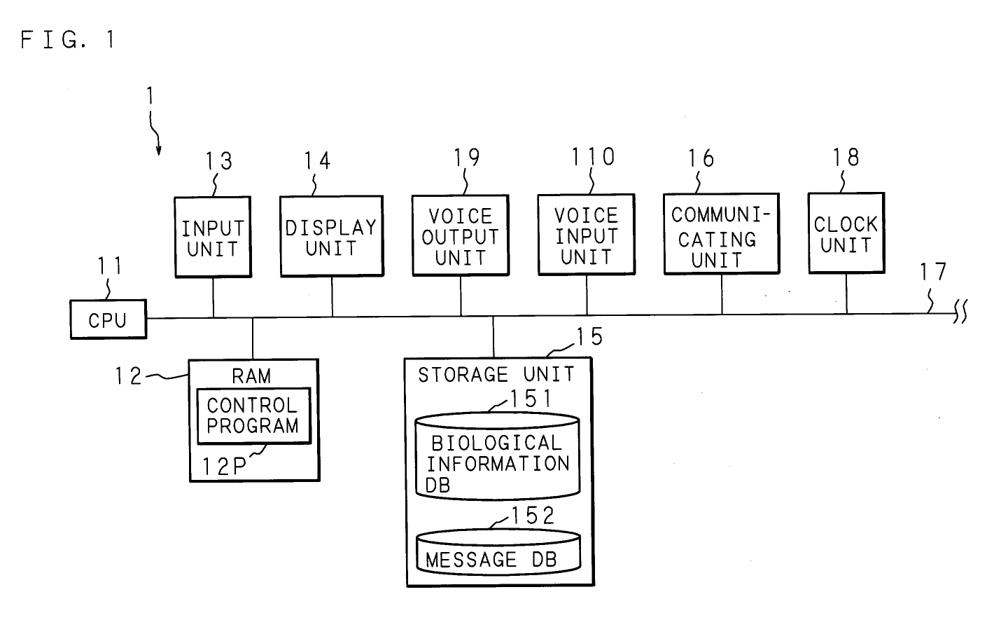

FIG. 1 is a block diagram showing the hardware structure of a biological information management apparatus. As shown in FIG. 1, connected through a bus 17 to a CPU (Central Processing Unit, processor) 11 of a biological information processing apparatus 1 are a RAM (Random Access Memory) 12; a storage unit 15 such as a hard disk; a communicating unit 16 such as a LAN (Local Area Network) card for transmitting and receiving information (biological information) to/from a server computer placed in a hospital, etc.; a clock unit 18 for outputting date information; a display unit 14 such as a touch panel display; and an input unit 13. A control program 12P for controlling the storage unit 15, display unit 14, input unit 13, etc. is stored in the RAM 12.

The storage unit 15 includes a biological information database (hereinafter referred to as the “biological information DB”) 151 that stores biological information such as blood pressure, according to a date on which the biological information was inputted, and a message database 152 that stores a plurality of pieces of message data classified into a plurality of categories. Moreover, generally appropriate values for biological information are stored as the third criteria in the storage unit 15. Examples of the stored third criteria are 110 to 130 (mmHg) for systolic blood pressure; 60 to 80 (mmHg) for diastolic blood pressure; ±3 Kg of a value obtained by squaring the initially registered height of a person cared for, further multiplying the resultant value by 22 and then dividing the resultant value by 1000 for weight; and pre-registered normal temperature of the person cared for (for example, from 35.5° C. to 36.7° C.) for body temperature, and 65 to 100 (mg/dl) for blood pressure. Note that it is possible to input suitable values as the third criteria from the input unit 13, or the third criteria may be downloaded from a server computer of a hospital (not shown) through the communicating unit 16.

In addition, the biological information processing apparatus comprises a voice output unit 19 as a message output unit, such as a speaker, for outputting a message read from the message DB 152 as a sound wave representing voice information, and a voice input unit 110 such as a microphone for converting a sound wave into an electrical signal. Furthermore, as the message output unit, the display unit 14 such as a liquid crystal display for outputting a message read from the message DB 152 as character information is provided. Note that messages can be outputted by voice, characters or the like as described above, but, to simplify the explanation, it is supposed in the following explanation that the messages are voice. Hereinafter, the messages are referred to as voice, the message output unit is referred to as the voice output unit 19, and the message DB is referred to as the voice DB 152.

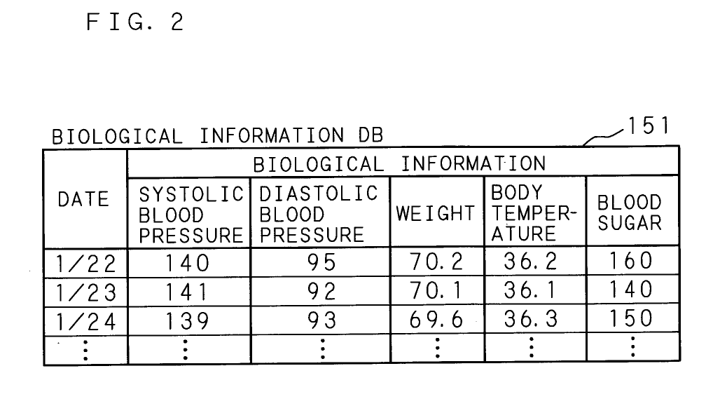

FIG. 2 is an explanatory view showing the record layout of the biological information DB 151. As shown in FIG. 2, the biological information DB 151 has a relational database structure, and the CPU 11 executes the processes of storing and searching biological information, etc. with respect to the biological information DB 151 by using an SQL (Structured Query Language). Biological information such as the systolic blood pressure is stored in accordance with the inputted month, day and time of the biological information. The data about month and day is stored based on the date data supplied from the clock unit 18. The biological information is stored by inputting it directly from the input unit 13 by the user, or may be stored based on the outputs from various measuring devices (not shown) connected through the communicating unit 16. Note that the biological information is not limited to those mentioned above, and may include other biological information such as heart rates.

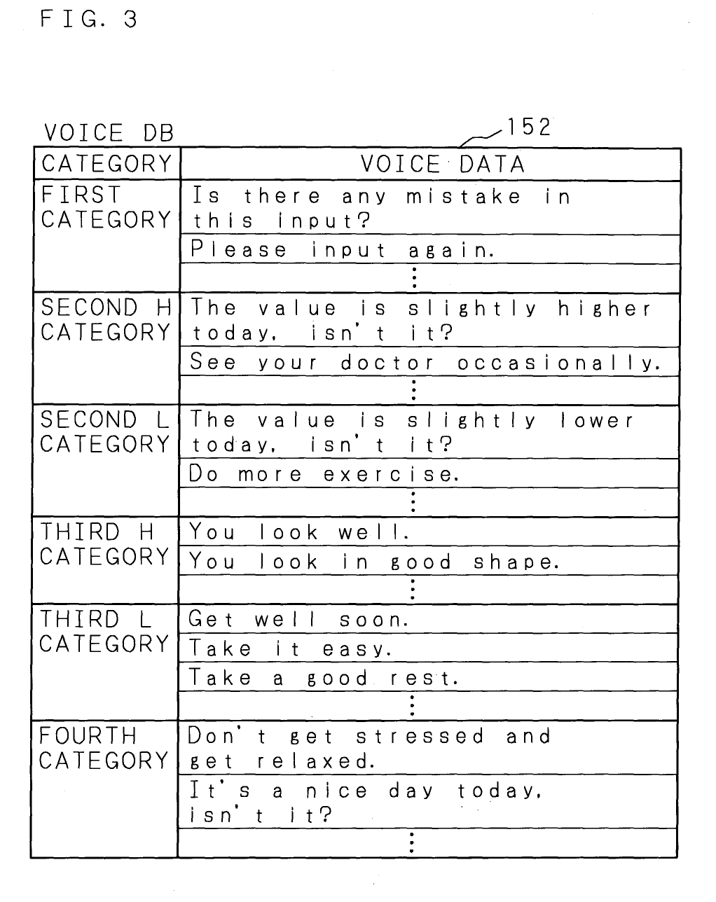

FIG. 3 is an explanatory view showing the record layout of the voice DB 152. Voice data to be outputted are classified into a plurality of different categories and stored in a WAV file format, etc. Note that, when a message is character data, text data corresponding to voice data is stored. In order to change the voice to be outputted according to the values of biological information, the voice data is classified into a plurality of categories. The following description will explain each category in detail. In the first category, there are stored voice data which is to be outputted when the inputted biological information does not meet the first criteria, and, for example, “Is there any mistake in this input?” is stored.

The first criteria are calculated based on the past biological information stored in the biological information database 151 (for example, the average value of the last ten days). For example, when the average value of the systolic blood pressure in the last ten days is 100 mmHg, this average value is multiplied by 0.7 or 1.3, so that values not less than 70 mmHg but not more than 130 mmHg are given (if the first criteria are set at ±30% of the average value). Note that these pieces of voice data are classified into a plurality of categories and stored in advance. Moreover, these voice data and categories may be downloaded from a server computer (not shown) of a hospital through the communicating unit 16.

In the second H category or the second 2L category, there are stored voice data which is to be outputted when the inputted biological information meets the first criteria, but does not meet the second criteria. For example, voice data such as “The value is slightly higher today isn't it?” or “The value is slightly lower today, isn't it?” is stored. The second criteria are calculated based on the past biological information stored in the biological information database (for example, the average value of the last ten days) and the first criteria. For example, when the average value of systolic blood pressure in the last ten days is 100 mmHg, this average value is multiplied by 0.8 or 1.2, so that values not less than 80 mmHg but not more than 120 mmHg are stored (if the second criteria are set at ±20% of the average value). Then, the second criteria are set by considering the first criteria so that the second criteria fall within the range of the first criteria.

Further, when the inputted biological information does not meet the second criteria and exceeds the upper limit of the second criteria (exceeds 120 mmHg in the above-mentioned example), the corresponding category is the second H category. When the inputted biological information does not meet the second criteria and is less than the lower limit of the second criteria (less than 80 mmHg in the above-mentioned example), the corresponding category is the second L category.

In the third H category or the third L category, there are stored voice data which is to be outputted when the inputted biological information does not meet the third criteria that are appropriate values pre-stored in the storage unit 15 and the change in the biological information satisfies a certain condition. For example, when the third criteria for systolic blood pressure are values between 110 mmHg and 130 mmHg, the systolic blood pressure of the person cared for is approaching the third criteria continuously such that 148 mmHg→145 mmHg→140 mmHg since three days ago, and then the third H category is selected and voice data respecting the effort for the treatment, such as “You look well” is outputted, although the inputted biological information does not satisfy the third criteria.

On the other hand, when the value is deviating away from the third criteria such that 140 mmHg→145 mmHg→148 mmHg, then the third L category is selected, and voice data worrying about the gradual deviation from the third criteria, such as “Take it easy” is outputted. In the fourth category, there is stored suitable voice data which does not particularly relate to the above-mentioned reasons, for example, various kinds of voice data such as “Don't get stressed and get relaxed” and “It's a nice day today, isn't it”.

In accordance with the above-described structures, the following description will explain the processing procedure of the present invention with reference to the flowchart.

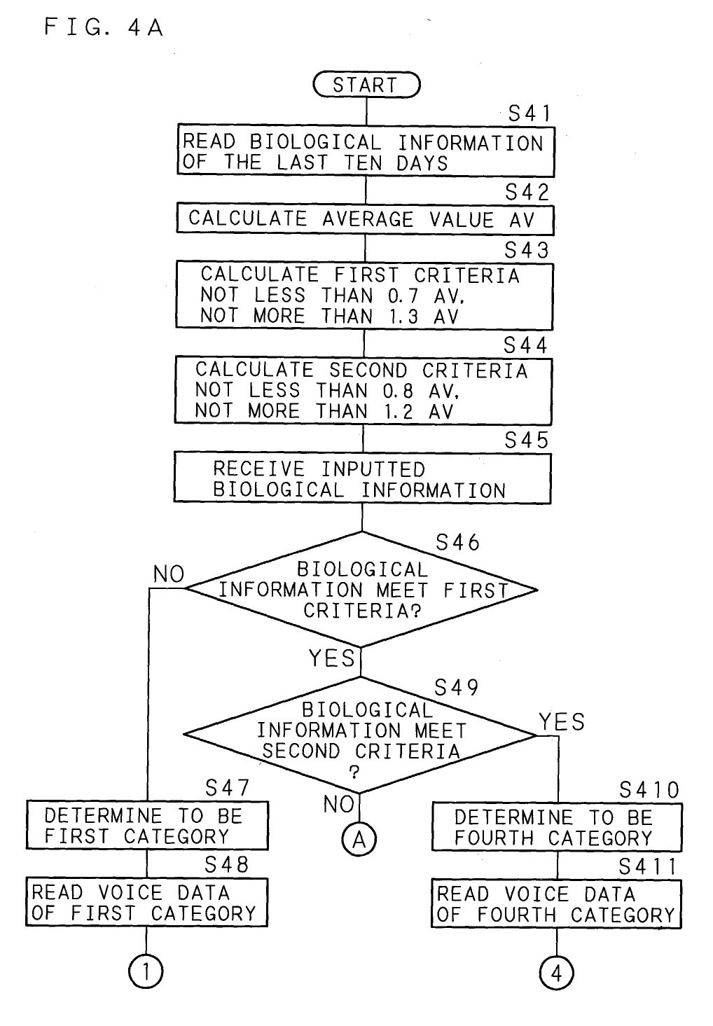

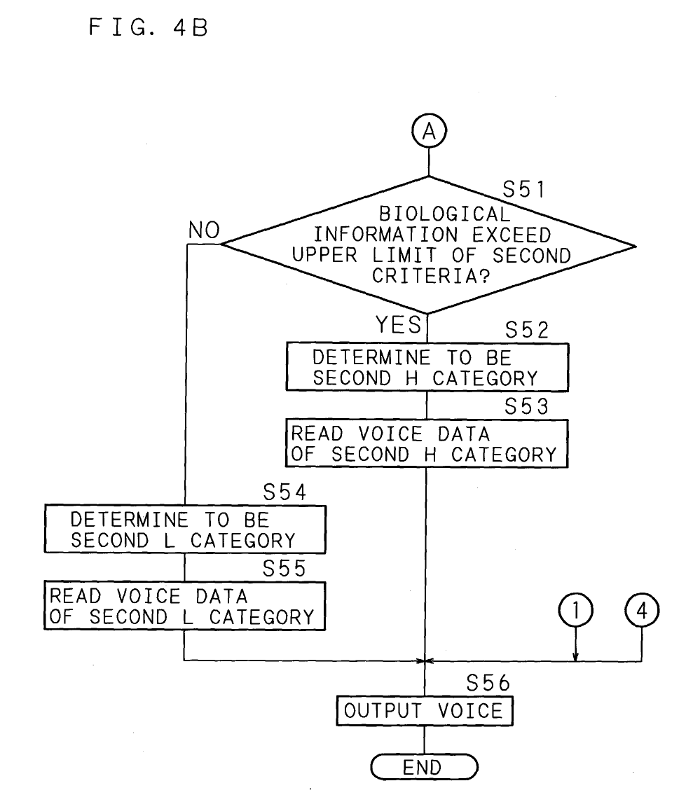

FIG. 4A and FIG. 4B illustrate the flowchart showing the processing procedure for determining the first criteria and the second criteria. First, the CPU 11 refers to the biological information DB 151 and reads biological information of the last ten days (step S41), and then calculates the average value AV of each piece of the biological information such as the blood pressure (step S42). Subsequently, the first criteria are calculated by multiplying the average value by predetermined factors (step S43). Here, the factors are set to 0.7 and 1.3, for example. Accordingly, the first criteria are not less than 0.7 AV, but not more than 1.3 AV.

Next, the second criteria are calculated (step S44). The second criteria need to be different from the first criteria, and, for example, the factors are set to 0.8 and 1.2 here. Accordingly, when the average value of the biological information of the last ten days is denoted as AV, the second criteria are not less than 0.8 AV but not more than 1.2 AV. When a person cared for inputs biological information from the input unit 13, the CPU 11 receives the inputted biological information (step S45).

The CPU 11 judges whether or not the inputted biological information meets the first criteria (step S46). More specifically, the CPU 11 judges whether the biological information is not less than 0.7 AV but not more than 1.3 AV. If the biological information does not meet the first criteria, the CPU 11 judges that a clearly erroneous value was inputted and determines that a category of the voice DB 152 to be read is the first category (step S47), and then reads arbitrary voice data belonging to the first category (step S48). On the other hand, if the biological information meets the first criteria, the CPU 11 skips steps S47 and S48, and moves to the next step.

The CPU 11 judges whether or not the inputted biological information meets the second criteria (step S49). More specifically, the CPU 11 judges whether the biological information is not less than 0.8 AV but not more than 1.2 AV. If the biological information meets the second criteria (YES in step S49), the CPU 11 judges that there is no particular problem and determines that a category of the voice DB 152 to be outputted is the fourth category (step S410), and then reads arbitrary voice data belonging to the fourth category (step S411). On the other hand, if the biological information does not meet the second criteria, the CPU 11 skips steps S410 and S411, and moves to the next step.

Thereafter, the CPU 11 judges whether or not the inputted biological information exceeds the upper limit of the second criteria (step S51). If the inputted biological information exceeds the upper limit (YES in step S51), more specifically, if the biological information is larger than 1.2 AV and smaller than 1.3 AV the CPU 11 determines that a category to be outputted is the second H category (step S52). Then, the CPU 11 reads arbitrary voice data belonging to the second H category (step S53). On the other hand, if the inputted biological information does not exceed the upper limit (NO in step S51), more specifically, if the biological information is smaller than 0.8 AV and larger than 0.7 AV, the CPU 11 determines that a category to be outputted is the second L category (step S54). Then, the CPU 11 reads arbitrary voice data belonging to the second L category (step S55). Finally, by outputting the voice data determined in each category to the voice output unit 19 (step S56), an optimum voice according to the inputted biological information is transmitted to the person cared for.

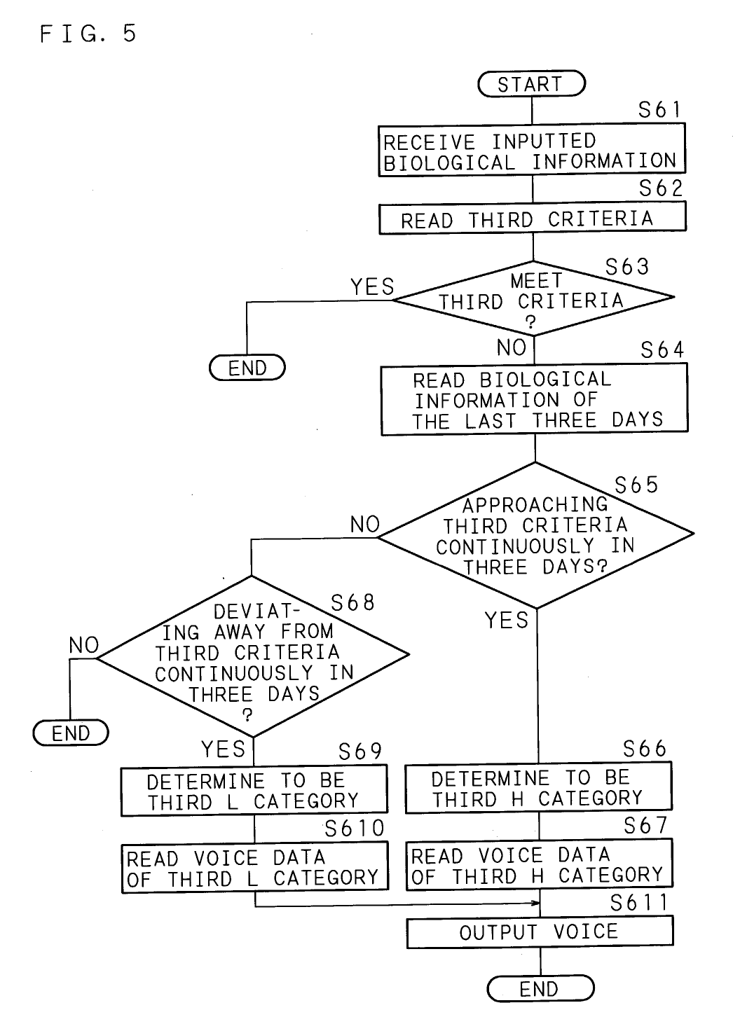

FIG. 5 is a flowchart showing the processing procedure to be followed when the third criteria are not met. When the person cared for inputs biological information from the input unit 13, the CPU 11 receives the inputted biological information (step S61). Subsequently, the CPU 11 reads the third criteria pre-stored in the storage unit 15 (step S62). Then, the CPU 11 judges whether or not the inputted biological information meets the third criteria (step S63). If the biological information meets the third criteria (YES in step S63), the CPU 11 completes the process without particularly giving a voice because the person cared for is in good condition. On the other hand, if the inputted biological information does not meet the third criteria (NO in step S63), the CPU 11 reads the biological information of the last three days from the biological information DB 151 (step S64).

Then, the CPU 11 calculates a change in the biological information. More specifically, the CPU 11 judges whether or not the biological information of the last three days is continuously approaching the third criteria (step S65). If the biological information is continuously approaching the third criteria (YES in step S65), the CPU 11 determines that a category to be outputted is the third H category (step S66). Then, the CPU 11 reads arbitrary voice data belonging to the third H category (step S67). On the other hand, if the biological information is not approaching the third criteria continuously in the three days (NO in step S65), the CPU 11 judges whether or not the biological information is deviating away from the third criteria continuously in the three days (step S68).

If the biological information is not deviating away from the third criteria continuously in the three days (NO in step S68), the CPU 11 completes the process without particularly giving a voice. On the other hand, if the biological information is deviating away from the third criteria continuously in the three days (YES in step S68), the CPU 11 determines that a category to be outputted is the third L category (step S69). Then, the CPU 11 reads arbitrary voice data belonging to the third L category (step S610). Finally, the CPU 11 outputs the voice data determined in each category to the voice output unit 19 (step S611). Thus, by causing the biological information management apparatus to give words that approves the effect of treatment on the patient, it is possible to significantly reduce the mental burden of the person care for, caused by the treatment.

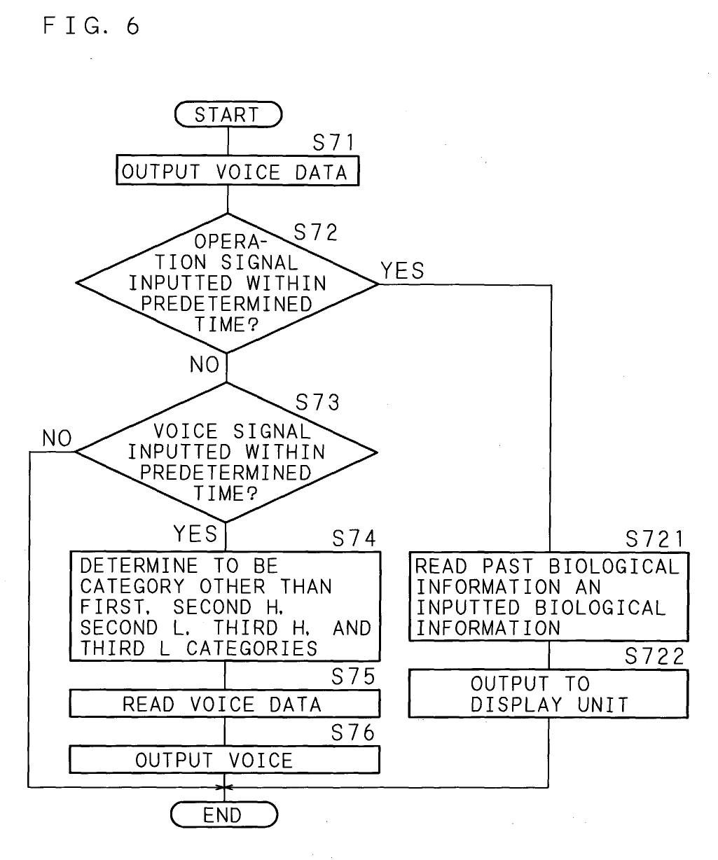

FIG. 6 is a flowchart showing the processing procedure after outputting voice data from the voice output unit 19. When voice data was outputted from the voice output unit 19 in steps S56 and S611 (step S71), the CPU 11 judges whether or not an operation signal was inputted from the input unit 13 within a predetermined time (for example, within 10 seconds) (step S72). If the operation signal was inputted (YES in step S72), the past biological information and the inputted biological information are read from the biological information DB 151 (step S721). Then, the past biological information and the inputted biological information are outputted to the display unit 14 (step S722). Note that the items to be outputted are not necessarily limited to those mentioned above, and an average value of the last few days, etc. may be outputted. Accordingly, it is possible to give interesting information immediately to the person cared for, who responded quickly to the voice output of the biological information management apparatus 1.

On the other hand, if an operation signal was not inputted within the predetermined time (NO in step S72), the CPU judges whether or not a voice signal was inputted within the predetermined time (step S73). More specifically, the CPU 11 stores the voice inputted from the voice input unit 110 in the RAM 12, and judges whether or not voice exceeding a certain threshold value was inputted. If a voice signal was not inputted within the predetermined time (NO in step S73), the CPU 11 completes the process without outputting voice data because the person cared for did not particularly respond. Note that, after the biological information management apparatus 1 gave words in step S71, the person cared for may give a voice to reply to the words, and examples of such a voice include a doubtful voice (really ?), convinced voice (Yes), agreeable voice (I see), surprised voice, and discouraged voice.

On the other hand, if a voice signal was inputted from the voice input unit 110 within the predetermined time (YES in step S73), the CPU 11 determines that a category of voice data to be outputted from the voice DB 152 is the fourth category other than the above-described first and second H, second L, third H and third L categories (step S74). Then, the CPU 11 reads arbitrary voice data belonging to the fourth category (step S75). Finally, the CPU 11 outputs the read voice data to the voice output unit 19 (step S76). Thus, it is possible to give sympathetic words to the person cared for, who responded quickly to the voice output of the biological information management apparatus 1, and significantly reduce the mental burden of the person cared for.

Second Embodiment

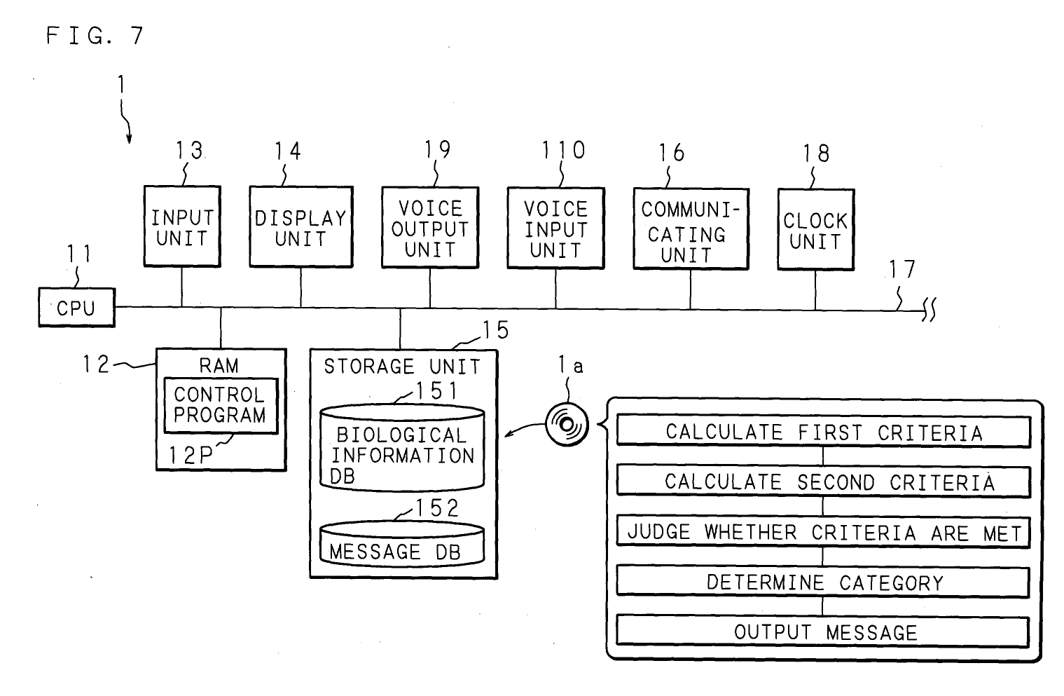

FIG. 7 is a schematic view showing the abstract of the second embodiment of the present invention. A computer program for implementing the biological information management apparatus 1 according to the first embodiment can be provided by being preinstalled in the biological information management apparatus 1 as in the second embodiment, or provided as a removable memory product such as a CD-ROM and MO. It is also possible to provide the computer program by transmitting it as a carrier wave via a channel. The content thereof is explained below.

A memory product 1a (such as a CD-ROM, MO or DVD-ROM) storing a program, which causes the biological information management apparatus 1 shown in FIG. 7 to calculate the first criteria and the second criteria, judge whether or not the criteria are met, determine a category and output a message, is installed in the storage unit 15 of the biological information management apparatus 1. This program is executed by loading it in the RAM 12 of the biological information management apparatus 1. Thus, the program functions as the biological information management apparatus 1 of the present invention as described above.

The second embodiment has the above-described structures. Since other structures and functions of the second embodiment are the same as those in the first embodiment, the corresponding parts are designated with the same reference numbers, and the detailed explanation thereof is omitted.

As this invention may be embodied in several forms without departing from the spirit of essential characteristics thereof, the present embodiment is therefore illustrative and not restrictive, since the scope of the invention is defined by the appended claims rather than by the description preceding them, and all changes that fall within metes and bounds of the claims, or equivalence of such metes and bounds thereof are therefore intended to be embraced by the claims.

1. A biological information management apparatus for storing inputted biological information in a biological information database and outputting the stored biological information to the outside, comprising:
a message database storing message data classified into a plurality of categories;
a processor capable of performing the following operations, including
a step of calculating first criteria and second criteria which are different from the first criteria, based on past biological information stored in the biological information database,
a step of judging whether or not biological information inputted meets the calculated first criteria or second criteria by comparing the inputted biological information with the first criteria or the second criteria,
a first determining step of determining a category of message data if it is judged that the inputted biological information does not meet the first criteria, and
a second determining step of determining a category of message data different from the category determined in the first determining step if it is judged that the inputted biological information meets the first criteria,
but does not meet the second criteria; and
a message output unit for outputting message data belonging to the determined category.

2. The biological information management apparatus of claim 1,
wherein the processor is further capable of performing the operations of
a step of reading pre-stored third criteria,
a step of judging whether or not biological information inputted meets the read third criteria by comparing the inputted biological information with the third criteria,
a step of calculating a change in the biological information, based on the inputted biological information and the past biological information stored in the biological information database, if it is judged that the inputted biological information does not meet the third criteria, and
a third determining step of determining a category of message data, based on the calculated change.

3. The biological information management apparatus of claim 2, wherein the processor is further capable of performing the operations of
a step of judging whether or not a voice signal or an operation signal was inputted within a predetermined time after outputting message data belonging to the category determined in the first determining step, second determining step, or third determining step from said message output unit,
a step of outputting message data belonging to a predetermined category in the message database from said message output unit if it is judged that a voice signal was inputted, and
a step of outputting the biological information stored in the biological information database to the outside if it is judged that an operation signal was inputted.

4. A biological information management apparatus for storing inputted biological information in a biological information database and outputting the stored biological information to the outside, comprising:
a message database storing message data classified into a plurality of categories;
means for calculating first criteria and second criteria which are different from the first criteria, based on past biological information stored in the biological information database;
means for judging whether or not biological information inputted meets the calculated first criteria or second criteria by comparing the inputted biological information with the first criteria or the second criteria;
first determining means for determining a category of message data if it is judged that the inputted biological information does not meet the first criteria;
second determining means for determining a category of message data different from the category determined by said first determining means if it is judged that the inputted biological information meets the first criteria, but does not meet the second criteria; and
a message output unit for outputting message data belonging to the determined category.

5. The biological information management apparatus of claim 4, further comprising:
means for reading pre-stored third criteria;
means for judging whether or not biological information inputted meets the read third criteria by comparing the inputted biological information with the third criteria;
means for calculating a change in the biological information, based on the inputted biological information and the past biological information stored in the biological information database, if it is judged that the inputted biological information does not meet the third criteria; and
third determining means for determining a category of message data, based on the calculated change.

6. The biological information management apparatus of claim 5, further comprising:
means for judging whether or not a voice signal or an operation signal was inputted within a predetermined time after outputting message data belonging to the category determined by said first determining means, second determining means, or third determining means from said message output unit;
means for outputting message data belonging to a predetermined category in the message database from said message output unit if it is judged that a voice signal was inputted, and
means for outputting the biological information stored in the biological information database to the outside if it is judged that an operation signal was inputted.

7. The biological information management apparatus of claim 1,
wherein said message output unit is a voice output unit for outputting the message data as voice information, or a display unit for displaying the message data as character information.

8. The biological information management apparatus of claim 4,
wherein said message output unit is a voice output unit for outputting the message data as voice information, or a display unit for displaying the message data as character information.

9. A memory product storing a computer program for storing inputted biological information in a biological information database and outputting the stored biological information to the outside, said computer program comprising:
a step of causing a computer to calculate first criteria and second criteria which are different from the first criteria, based on past biological information stored in the biological information database;
a step of causing the computer to judge whether or not biological information inputted meets the calculated first criteria or second criteria by comparing the inputted biological information with the first criteria or the second criteria;
a first determining step of causing the computer to refer to a message database that stores message data classified into a plurality of categories and determine a category of message data if it is judged that the inputted biological information does not meet the first criteria;
a second determining step of causing the computer to determine a category of message data different from the category determined in the first determining step if it is judged that the inputted biological information meets the first criteria, but does not meet the second criteria; and
a message outputting step for causing the computer to output message data belonging to the determined category.

10. The memory product of claim 9, wherein said computer program further comprises the steps of:
causing the computer to read pre-stored third criteria;
causing the computer to judge whether or not biological information inputted meets the read third criteria by comparing the inputted biological information with the third criteria;
causing the computer to calculate a change in the biological information, based on the inputted biological information and the past biological information stored in the biological information database, if it is judged that the inputted biological information does not meet the third criteria; and
a third determining step of causing the computer to determine a category of message data, based on the calculated change.
